(19) United States
(12) Patent Application Publication
(10) Pub. No.: US 2010/0145049 A1
(43) Pub. Date: Jun. 10, 2010

(54) PROCESS FOR MAKING N-(DIPHENYLMETHYL)PIPERAZINES

(76) Inventors: Jie Zhu, Nijmegen (NL); Judith Janneke Firet, Ede (NL)

Correspondence Address: SYNTHON IP INC, P.O. BOX 1364, FAIRFAX, VA, 22038-1364, US

(21) Appl. No.: 12/623,591

(22) Filed: Nov. 23, 2009

Related U.S. Application Data
(60) Provisional application No. 61/199,920, filed on Nov. 21, 2008.

(51) Int. Cl. C07D 241/04

(52) U.S. Cl. 544/389; 544/396

(57) ABSTRACT

A compound of formula (8) or a salt thereof:

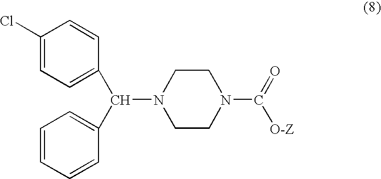

wherein Z represents a group containing 1-20 carbon atoms and at least one chiral carbon atom and having a single conformation, is useful in the synthesis of pharmaceutical compounds, especially chiral compounds such as levocetirizine.

This application claims the benefit of priority under 35 U.S.C. §119(e) from earlier filed U.S. Provisional Application Ser. No. 61/199,920, filed Nov. 21, 2008, the entire contents of which are incorporated herein by reference.

Cetirizine, chemically 2-[4-[(4-chlorophenyl)-phenyl-methyl]piperazin-1-yl]ethoxy]acetic acid, is a useful pharmaceutical active ingredient. It is an antihistamine whose principal effects are mediated via selective inhibition of H₁ receptors. This anti-allergy drug is marketed by the company UCB (which is also the originator of the drug) and/or Pfizer under the brand name Zyrtec®, as a dihydrochloride salt (often referred to as “cetirizine hydrochloride”) as shown below.

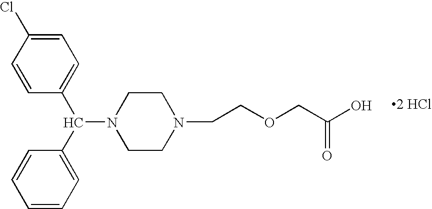

The drug is indicated for the relief of symptoms associated with seasonal allergic rhinitis or perennial allergic rhinitis, as well as for the treatment of the uncomplicated skin manifestations of chronic idiopathic urticaria in adults and children 6 months of age and older.

Cetirizine has one asymmetric carbon, therefore it may be resolved into enantiomers. The pharmaceutically active enantiomer in the racemic cetirizine is the levocetirizine, which is the (R) enantiomer of cetirizine. A medicament comprising levocetirizine was launched in the first quarter of 2001 in Germany followed by a pan-European launch. Levocetirizine is also marketed as the dihydrochloride salt, under the brand name Xyzaal®.

Cetirizine was disclosed in U.S. Pat. No. 4,525,358 (EP 58146). Levocetirizine was specifically disclosed in GB2225321. The method of use of levocetirizine has been disclosed in U.S. Pat. No. 5,698,558 (EP 663828).

Conventionally, levocetirizine may be obtained by resolution of the cetirizine enantiomers as generally suggested, e.g., in WO 94/06429. However, the effectiveness of such a process is apparently not high and therefore it is preferred to make levocetirizine from an enantiopure intermediate.

One such useful intermediate is the compound of formula (4).

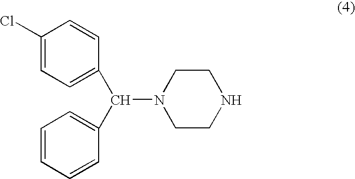

The presence of a quaternary carbon in the formula (4) indicates that the compound may be obtained as a racemate or as a single enantiomer, particularly as the (R) enantiomer. This intermediate may be converted to cetirizine or related analogues, particularly to racemic cetirizine or levocetirizine, by various known processes, e.g., by processes reviewed in U.S. Pat. No. 4,525,358. Resolution of the intermediate (4) into enantiomers by L-tartaric acid as well as the process for making levocetirizine from the corresponding enantiomer of (4) was disclosed in GB2225321. However, the yield and effectiveness of the resolution is insufficient, as shown in U.S. Pat. No. 5,478,941.

The useful starting material for making the compound (4) is the well known and commercially available compound of formula (1),

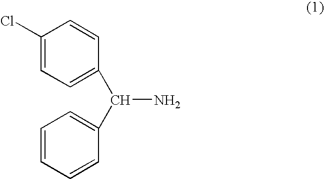

Similarly as the above compound (4), the compound (1) may be obtained as a racemate or as a single enantiomer, particularly as the (R) enantiomer. It is known that the racemic compound (1) can be easily and effectively resolved into enantiomers by a fractional crystallization, preferably by the crystallization of salts with L-tartaric acid. (see U.S. Pat. No. 5,478,941). This makes the compound (1) an important intermediate, particularly in the synthesis of an enantiomerically pure (4).

In a known process for making compound (4) disclosed in EP 617028 (U.S. Pat. No. 5,478,941), the racemic compound of formula (1) and/or its (R)-enantiomer is subjected to a condensation with the N-sulfonated bis-chloroethylamine compound of formula (2),

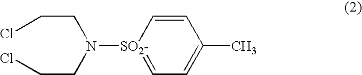

to form the compound of formula (3).

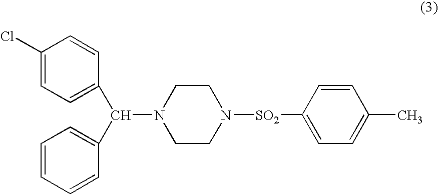

The compound (3) is then deprotected to form the key intermediate of general formula (4). A disadvantage, however, with the use of the compound of formula (2) in the synthesis of the compound of formula (4) is the need to use a strong deprotecting agent. The tosyl-protective group may be effectively removed only by using a solution of hydrogen bromide in acetic acid. This agent is extremely corrosive, irritating, and toxic so that special measures must be used in employing this material.

In principle, one could expect that also an unprotected compound of the formula (5a)

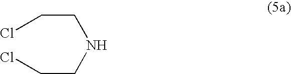

might be used for coupling with the compound (1). This would avoid the deprotection step and form the compound (4) directly. But this option is not satisfactory. First, the compound (5a) is an extremely toxic compound (“mustard gas”), and second the reaction is accompanied with a large amount of side products arising particularly from the self-condensation of the compound (5). Thus, the use of an N-protected bis-haloethylamine is clearly preferable. But other potentially useful N-protected compounds, e.g. a carbonyl, alkyl or a triphenylmethyl protecting group, have been reported as unsatisfactory. U.S. Pat. No. 5,478,941 and EP 955295 teach that the above mentioned N-tosyl compound of formula (2) is the only useful compound for the coupling reaction with (1). The protected analogues (a carbonyl, alkyl, or trityl protecting group) caused important racemization of the compound (1) during the coupling reaction and/or the formation of undesired by-products.

Opalka C. J. et al. (Synthesis 1995 (7), p. 766-768) reports that the coupling reaction failed if the amides of formula 6, wherein R represents a carbon-terminated substituent, were used.

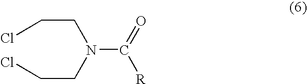

Thus other protecting groups have proven to be unsuitable so far. It would be desirable to have an alternative process for making the compound of general formula (A), particularly for making the R-enantiomer thereof, the levocetirizine.

### SUMMARY OF THE INVENTION

The present invention relates to the discovery of a convenient process for making levocetirizine, and intermediates useful therein, from achiral precursors. Accordingly a first aspect of the invention relates to a compound of formula (8) or a salt thereof:

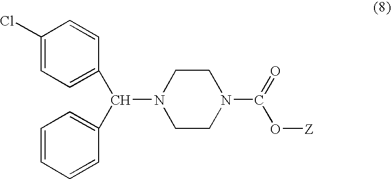

wherein Z represents a group containing 1-20 carbon atoms and including at least one chiral carbon atom and having a single conformation. Typically Z is a substituted or unsubstituted alkyl group, cycloalkyl group, aralkyl group, or alkylaryl group, wherein the substituents are selected from alkyl, cycloalkyl, halogen, alkoxy, amino, and/or nitro groups. Outside of the Z group, the chiral methyl, i.e., the methyl bridging the two phenyl groups, can have both conformations (i.e., a mixture of (R) and (S)), or may be a single conformation. When present as a mixture of conformations, the compounds of formula (8) represent a pair of diastereomers. The pair of diastereomers can be resolved, even without the use of an optically active salt, into the single diastereomer and are thus useful intermediates in forming single enantiomers of N-dipenylmethyl-piperazine compounds especially levocetirizine.

Hence another aspect of the invention relates to a process, which comprises:
(a) providing a pair of diastereomers of the formula (8) or a salt thereof:

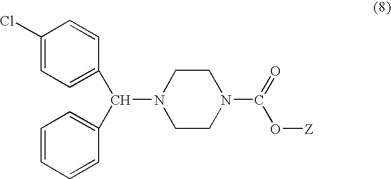

wherein Z represents a group containing 1-20 carbon atoms and at least one chiral carbon atom and having a single conformation;
(b) resolving said diastereomers of formula (8) to obtain the single diastereomer having the methyl carbon in the R-conformation; and
(c) hydrolyzing the single diastereomer of the compound (8) having the methyl carbon in the R-conformation to form the (R)-enantiomer of the compound (4)

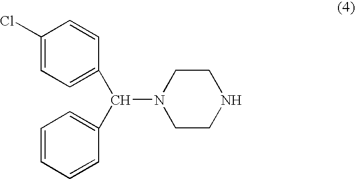

or a salt thereof. Having the (R)-conformation of the compound of formula (4) allows for the convenient conversion thereof to levocetirizine, such as by the methods described above.

### DETAILED DESCRIPTION OF THE INVENTION

The present invention deals with an alternate process for making the single, preferably (R), enantiomer of a compound of formula (4), which is the key intermediate in the synthesis of pharmaceutically useful compounds and in particular levocetirizine. The process is characterized by using an optically active substituent, which allows for the resolution of the intermediates into single enantiomers without the need to use an optically active acid. As any process of making levocetirizine via the intermediate (4) requires the use of optically active acids as resolution agents, the finding that such acids can be avoided is surprising. And in as much as the optically active substituent is preferably used in the synthesis of compound (4), the process can essentially serve two functions at once.

As used herein, the term “chiral methyl group” refers to the chiral carbon bridging the two phenyl groups as shown in the compounds (1), (4) and (8). This chiral methyl group is outside of the group Z and should not be confused with the “at least one chiral carbon” provided in the Z group. All chemical formulas herein having a chiral carbon present include both mixtures of the enantiomers such as a racemate as well as a single enantiomer, unless noted otherwise. Also, as used herein a “single” or “pure” isomer, enantiomer, or diastereomer does not require absolute purity from the corresponding conformational pair, but rather means that the compound possess at least 90% isomer/enantiomer/diastereomeric purity, preferably at least 95% purity, and in some embodiments including at least 98% and at least 99% purity. Similarly, all chemical formulas, e.g., (1) to (8), include the acid addition salts thereof unless explicitly stated to the contrary.

In the first step of the process of the present invention, a compound of the formula (8) is provided as a pair of diastereomers.

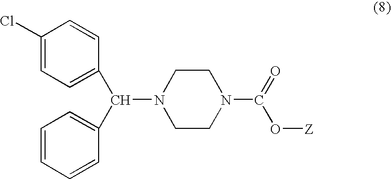

The compound of the formula (8) comprises a chiral group Z that contains 1-20 carbon atoms and at least one chiral carbon atom and having a single conformation. The single conformation of the chiral substituent, i.e., only the (R) conformation, in combination with the chiral methyl group, enables the formation of diastereomers. The single conformation of the group Z is thus a rigid orientation. On the other hand, the chiral methyl group (the carbon bridging the phenyl groups) may have random orientation of substituents, either R or S. Hence, the compound (8) can be a pair of diastereomers; e.g., if the group Z comprises one chiral carbon with the orientation (R), then the pair of diastereomers of the compound (8) have the conformation (R,R) and (S,R), respectively. Typically the group Z represents a C1-C20 substituted or unsubstituted alkyl group, cycloalkyl group, aralkyl group, or alkylaryl group, wherein the substituents are selected from alkyl, cycloalkyl, halogen, alkoxy, amino, and/or nitro groups. In some embodiments Z is a C6 to C20 substituted cycloalkyl group.

An advantageous example of the group Z is a menthyl group (2-isopropyl- 5-methylcyclohexyl group). This group has three chiral carbons allowing for 8 stereoisomers; any rigid combination of spatial arrangement of substituents is allowable, provided however that the resulting conformation is rigid; i.e. always a single stereoisomer must be used. Advantageously, the menthyl group should have the same conformation as has the natural (−) menthol, i.e., (1R,2S,5R).

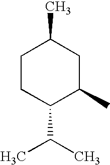

For convenience such a menthyl group will be denoted herein as (−)-menthyl group. Alternatively, the menthyl group may have the opposite conformation corresponding to (+)-menthol, i.e., (1S,2R,5S), such menthyl group will be denoted herein as (+)-menthyl group.

Accordingly, a preferred compound of formula (8), wherein Z is the (−)-menthyl group, is a compound of the formula (8a)

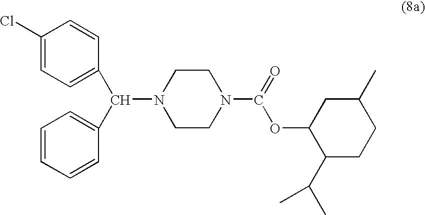

including a single diastereomer thereof, as well as a salt thereof, particularly the sulfate salt. Another example of a suitable group Z is a camphenyl group.

The compound (8) may be obtained by various ways. In a first process, the compound (8) is obtained by a reaction of the racemic compound (4) with a haloformate of the formula (9)

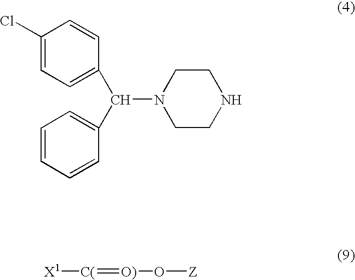

wherein X¹ is a halo group such as chloro or bromo group; and preferably X¹ is a chloro group; and Z is as defined above. Advantageously, the Z represents a single stereoisomer of a menthyl group, particularly (−)menthyl group, and X represents chlorine. Thus, the preferred compound for the reaction with the compound (4) is a menthyl chloroformate of the formula (9a).

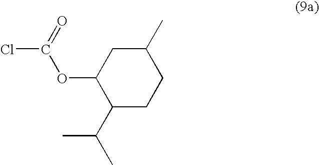

The reaction between compounds (4) and (9) generally proceeds in an inert, preferebly water immiscible, solvent, e.g. in a hydrocarbon or a halogenated hydrocarbon, preferably under presence of a base, which may be advantageously an organic base, for instance a primary, secondary or tertiary amine. The reaction temperature may be ambient or close to ambient (0-50° C.).

The side product (a salt of the amine) is conventionally removed by an extraction by water and the product is optionally isolated from the organic layer, e.g. by a removal of the solvent. The crude product may be purified, if necessary, or may be used in the next step in the crude state. The compound (8) may also be isolated as an acid addition salt.

In a second process, the pair of diastereomers of formula (8) is provided from racemic or optically impure formula (1). In particular, the racemic compound of formula (1) reacts, generally in a liquid phase, with the compound of the general formula (7) to yield the compound of formula (8).

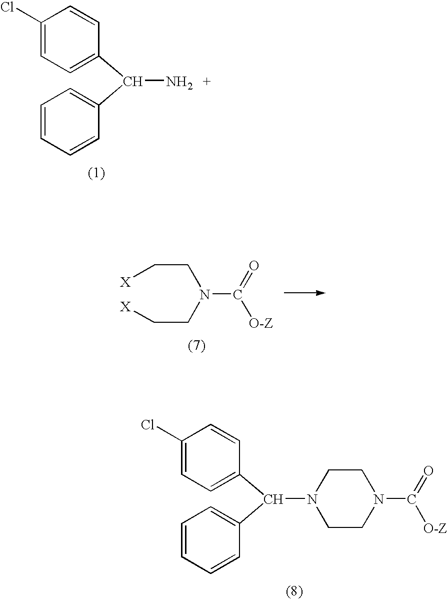

In the formulas (7) and (8), Z is the same group as defined above; preferably Z is a menthyl group.

The compound (7) contains two equal leaving groups X that are reactive with the primary amine in the compound (1) to form the piperazine ring. Such groups X may be represented by a halogen group, such as chloro or bromo group; or a sulfonyl group such as mesyloxy, besyloxy, anisylsulfonyloxy or tosyloxy group; preferably X is a chloro group.

Thus, the preferred example of the compound of the general formula (7) is the compound of formula (7a).

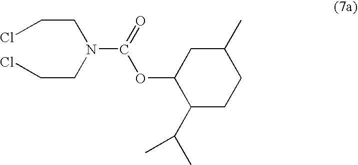

The reaction between compounds (1) and (7) proceeds in the presence of a base, which is preferably an organic base. In a convenient embodiment, a liquid organic base is employed, whereby the liquid organic base also serves as the solvent of the reaction. The preferred liquid organic base is diisopropylethylamine. The reaction preferably proceeds at an enhanced temperature, e.g., at a temperature between 50-150° C., suitably at reflux. Advantageously, potassium iodide may be added as an initiator. The reaction progress may be monitored by a suitable analytical technique, e.g. HPLC. After the reaction, the reaction mixture containing the product (8) may be used for the next step (advantageously, after removal of amine salts formed and/or after removal of at least part of the solvent) or is elaborated to isolate the reaction product (8). In a suitable way of isolation, the reaction mixture is partitioned between an aqueous and organic phase (whereby the organic solvent may be conveniently a hydrocarbon or a chlorinated hydrocarbon) and the product is isolated from the organic phase. The crude product may be purified, if necessary, or may be used in the next step in the crude state. The compound (8) may also be isolated as an acid addition salt.

If the preferred compound (7a) or (9a), respectively, is used, the reaction product is the compound of the formula (8a),

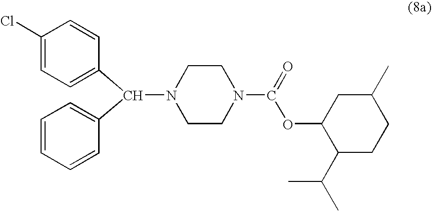

which comprises a pair of diastereomers differing in the orientation of substituents around the chiral methyl group.

In a second step, the pair of diastereomers of the compound (8) is resolved to obtain a single diastereomer having the chiral methyl group in the (R)-conformation; i.e., in the correct orientation for completing a levocetirizine synthesis. As a practical matter, each single diastereomer is usual obtained by the resolution process and thus either one could in fact be used in subsequent reactions; e.g., in forming other compounds that desire the (S)-conformation.

The compound (8), particularly the compound (8a), may be resolved into single diastereomers without the need of any additional resolution agent, i.e., without the need of an optically active acid. In practice, a suitable process comprises a fractional crystallization of the compound (8) from a suitable solvent. Another suitable process comprises chromatography on a suitable column, such as HPLC. Advantageously, but not necessarily, the compound (8) is first converted into a suitable acid addition salt before the fractional crystallization by contacting with a suitable acid in a suitable solvent, which may be the same or different from the solvent used for the crystallization. Examples of suitable acid addition salts are, without limitation, a hydrochloride, a hydrobromide, sulfate, phosphate, acetate, formate, maleate, fumarate, tartrate or oxalate, etc. Examples of suitable solvents for the fractional crystallization are, without limitation, water, an C1-C6 aliphatic alcohol, a C3-C8 aliphatic ketone, a C2-C8 aliphatic or cyclic ether, C2-C 10 ester, C1-C4 nitrile, and mixtures thereof.

In the fractional crystallization, one diastereomer of the compound (8) preferentially precipitates while the other preferentially remains in the solution. The word “preferentially” indicates that one diastereomer is more likely to precipitate under the crystallization conditions than the other, hence achieving a (partial) separation of the two diastereomers. The crystallization can be repeated one or more additional times under the same or different conditions until the desired purity (e.g. degree of separation) is achieved. Generally a single crystallization achieves at least about a 40% enrichment; e.g., starting from 50:50 mixture, a single crystallization provides at least about a 70:30 mixture. The conformation of the diastereomer in the precipitated/crystallized product depends on the choice of the group Z, on the solvent and on the nature of the compound (8), i.e. whether the compound (8) is a base or a salt. The solid product obtained by crystallization may be, but is not necessarily, the product with the desired (R)-conformation of the chiral methyl group. It may be isolated by filtration and optionally washed and dried. The other diastereomer that remained in the solution may be isolated as well, such as by evaporation of the solvent. Thus, whether the desired diastereomer is contained in the solid or the solvent, it can be isolated for use in subsequent reactions. If the isolated product has insufficient optical purity, the fractional crystallization of any of the obtained fractions may be repeated.

Typically, the preferred compound (8a) is fractionally crystallized as a base or is converted into a salt with sulfuric acid. If crystallized as a base, e.g. from an etheral solvent, the product with the desired (R) orientation preferentially remains in the solution. On the other hand, the sulfate salt crystallizes (e.g. from ethyl acetate and/or acetonitrile) as a solid preferentially with the (R) orientation, while the (S)-diastereomer is concentrated in the solution.

In the third step, the single diastereomer of the compound (8) is subjected to a hydrolysis/solvolysis of the carbamate group to form the single enantiomer of the compound of formula (4). The hydrolysis is advantageously performed by an aqueous or alcoholic acid or by an aqueous alkali. The acid may be, e.g., hydrochloric or sulfuric acid. The “aqueous alkali” comprises an aqueous solution or suspension of lithium, sodium, potassium or calcium hydroxide or carbonate. The reaction may proceed in the presence of an inert co-solvent. The reaction product comprising the single, preferably the (R)-enantiomer of the compound (4) is then advantageously extracted by a water-insoluble organic solvent, preferably by ethyl acetate and/or toluene, and isolated from the organic phase. Side products, if any, may be efficiently removed if the above extraction is done under acidic or alkaline conditions.

In an advantageous mode, the formed compound of formula (4) is isolated from the reaction mixture, and/or purified. It may be isolated as a free base or it may be isolated after converting it into an acid addition salt with an organic or inorganic acid that is isolatable as a solid, preferably crystalline, product. An advantageous salt in this respect is the oxalate salt as it may be isolated as a stable crystalline material. The oxalate salt of the compound (4) is a suitable form that allows storage of the compound (4), particularly the (R)-enantiomer thereof, for an enhanced period of time. The single enantiomer of the compound (4) may be however isolated also as a free base, which is preferably a solid product, for instance by a suitable extraction process. In an example, the reaction mixture is partitioned between an organic layer and acidified aqueous layer (in which the product concentrates), the aqueous layer is neutralized, the free base of (4) is extracted by an organic solvent and isolated from this solvent.

The starting (4-chlorophenyl)phenylmethylamine of formula (1) and the compound of formula (4) are known, commercially available compounds.

The compound of formula (7) may be obtained, for instance, by the condensation of the compound (5)

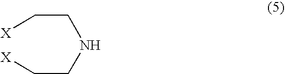

and/or an acid addition salt thereof, with a chloroformate compound of formula (9)

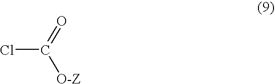

wherein X and Z have the above meaning. The preferred compound N,N-bis(2-chloroethyl) (−) menthyl carbamate of the formula (7a) is thus obtained by the reaction of the bis(2-chloroethyl)amine with a (−)menthylchloroformate of formula (9a).

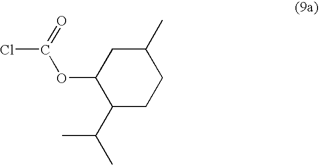

The reaction is advantageously performed in an inert solvent, e.g., in a hydrocarbon solvent or a halogenated hydrocarbon solvent, preferably in the presence of a base. Similarly, one may prepare N,N-bis(2-chloroethyl) (+) menthyl carbamate by the reaction of the bis(2-chloroethyl)amine with a (+)menthylchloroformate.

Alternatively, the compound of formula (7) may be obtained from bis (2-hydroxyethyl)amine and a haloformate (9) according to the scheme

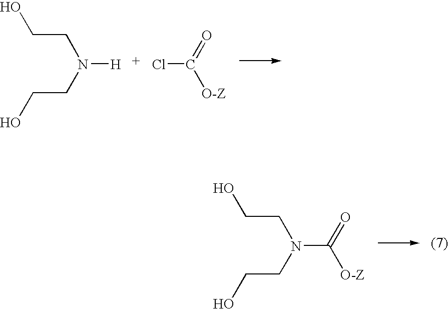

under general conditions known in the art.

The single (R)-enantiomer of the compound of formula (4), as well as acid addition salts thereof, prepared by the above process, may be converted into a levocetirizine compound by known means as described in the above cited patents.

The invention is illustrated by the following non-limiting examples.

### Example 1

### Preparation of Menthylcarbamate

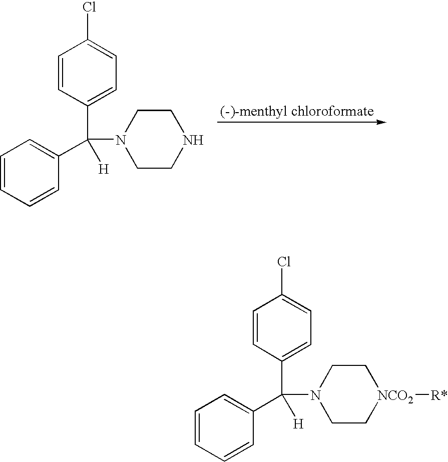

14.35 g (0.05 mol) piperazine derivative was dissolved in 100 ml dried dichloromethane, followed by addition of 15 ml (−)-menthyl chloroformate dropwise, while stirring at room temperature. The addition was completed within 15 minutes. 7 ml triethylamine was added in ˜2 minutes. Mixture was further stirred over night. 200 ml H₂O was added, and the mixture was stirred for another 30 minutes. Layers were separated. Water layer was extracted again with dichloromethane (25 ml). Combined organic layer was concentrated in vacuo to give a oily/semisolid material.

### Example 2

### Chiral Separation of Free Base

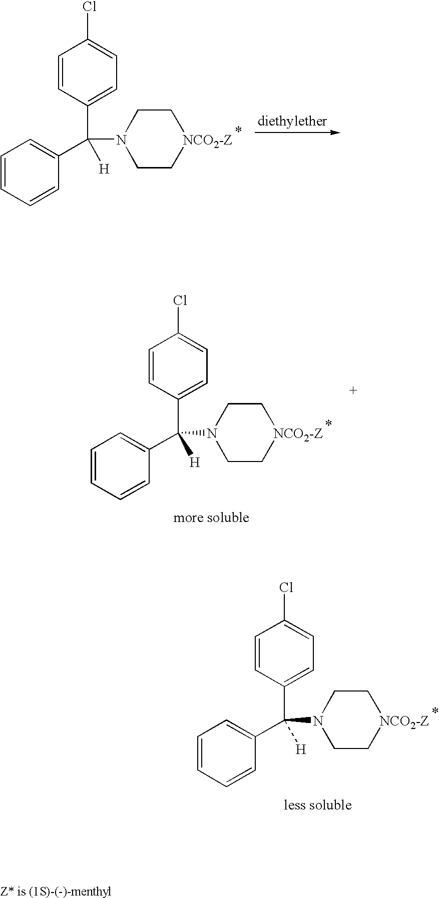

A mixture containing 7.62 g (±) carbamate in 30 ml diethyl ether were stirred at ˜4° C. for 2 hours. Solid was collected by filtration and dried. Enantiomeric enriched solid was suspended again in 7.5 ml diethyl ether and stirred for 2 hours at ˜4° C. The solid was isolated by filtration and dried. Enriched solid was suspended again in 5 ml diethyl ether and stirred for 6 hours at ˜4° C. The isolated carbamate compound (1.0 g) had a 97.5% in S enantiomeric purity.

### Example 3

### Chiral Separation of Free Base

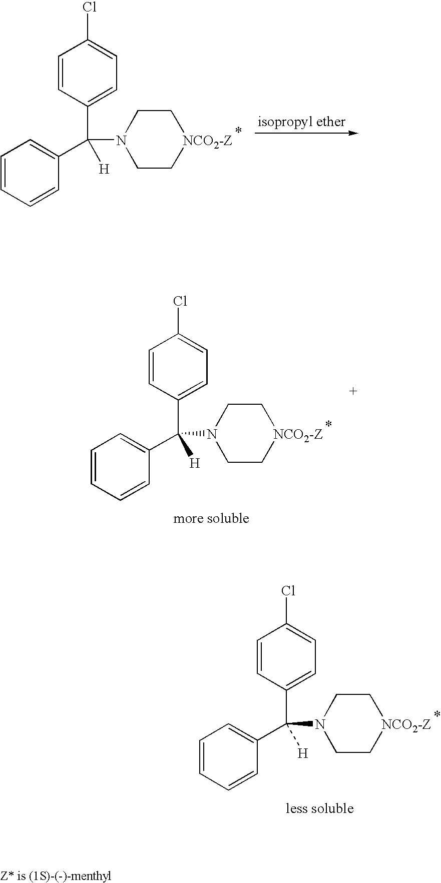

A mixture containing 5.48 g (±) Carbamate in 25 ml isopropyl ether were stirred at 40° C. for 30 minutes. Formed suspension was stirred at ambient temperature for 3 hours, and further at ˜4° C. overnight. Enriched solid was isolated by filtration and dried.

The isolated solid was suspended in 10 ml isopropyl ether. The suspension was stirred for 2 hours at 4° C. The solid was isolated again by filtration and dried.

Above procedure was repeated twice using 5 ml isopropyl ether. The isolated carbamate compound (1.36 g) had a 96% in S enantiomeric purity.

### Example 4

### Chiral Separation of Free Base

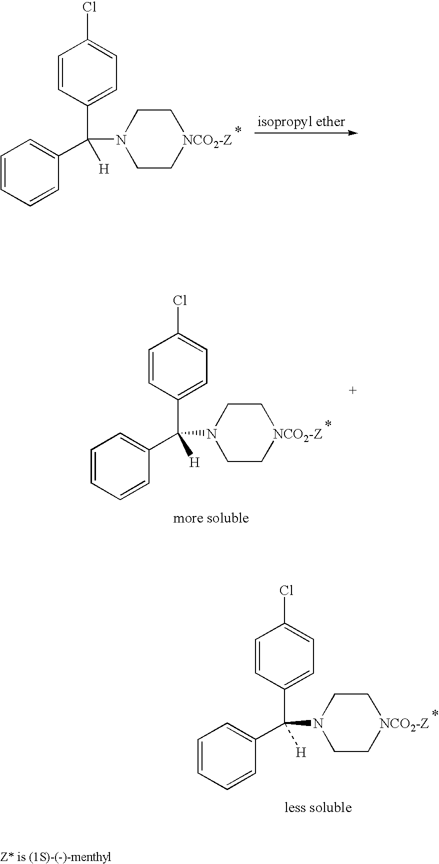

A mixture containing 11.84 g (±) Carbamate in 40 ml isopropyl ether were stirred at ambient temperature for 4 hours. Enriched solid was isolated by filtration and dried.

The isolated solid was suspended in 20 ml isopropyl ether. The suspension was stirred ambient temperature for 4 hours. The solid was isolated again by filtration and dried.

Above procedure was repeated once using 25 ml isopropyl ether. The isolated carbamate compound (2.62 g) had a 96.5% in S enantiomeric purity.

### Example 5

### Chiral Separation of Free Base

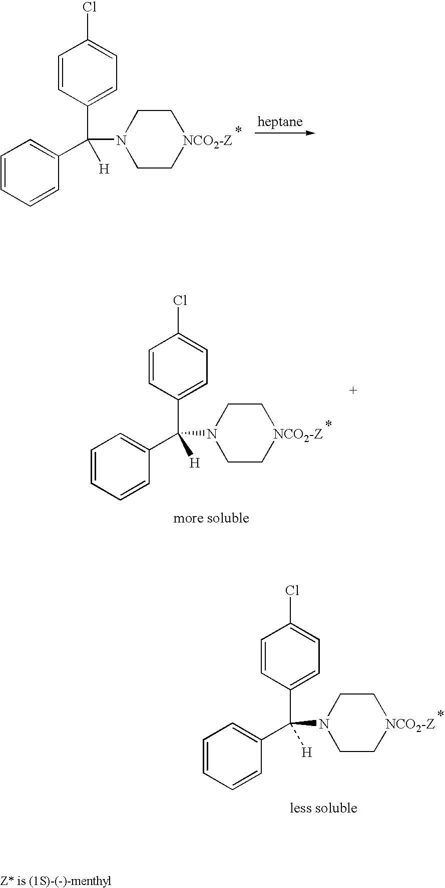

A mixture containing 3.91 g (±) Carbamate in 10 ml isopropyl ether was refluxed for 1 hour. Then it was stirred at ambient temperature for 4 hours. Solid was collected by filtration and dried. Enriched carbamate was suspended in 5 ml isopropyl ether. The suspension was refluxed for 1 hour. Then it was stirred at ambient temperature for 4 hours. Solid was collected by filtration and dried at 40° C. under vacuum. The isolated carbamate compound (1.22 g) had a 97.2% in S enantiomeric purity.

### Example 6

### Chiral Separation of Sulfuric Acid Salt

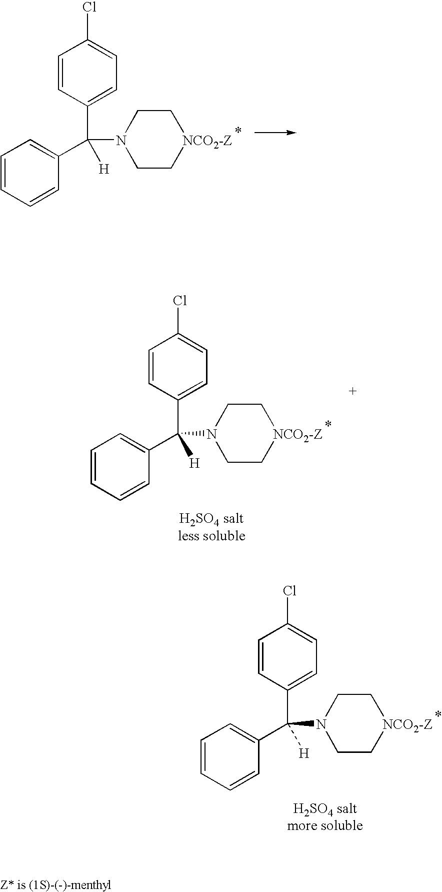

Crude (−) menthyl carbamate (prepared from 14.35 g piperazine derivative) was dissolved in 200 ml ethyl acetate. With stirring at room temperature, 4.9 g sulfuric acid was added dropwise in ˜2 minutes. Mixture was stirred for 2 hours. 25 ml acetonitrile was added, and the mixture was further stirred over night. Solid was filtered off, which showed an enantiomeric purity of ˜73%.

Crude solid was refluxed in a mixed solvent of 150 ml ethyl acetate and 25 ml acetonitrile for 1 hour and stirred at room temperature for 2 hours. Solid was collected by filtration. The washing process was repeated for 4 times. Final obtained solid showed >98% in R enantiomeric purity. 11.41 g solid was obtained after drying (˜40%).

### Example 7

### Hydrolysis of the Carbamate

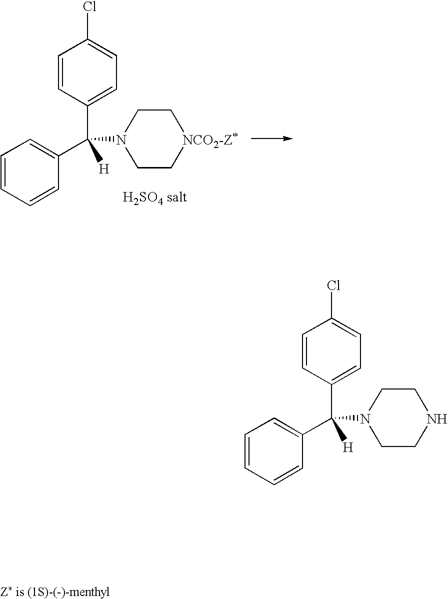

2.5 g carbamate salt was suspended in 5 ml isopropanol. With stirring at room temperature, 2 ml sulfuric acid was added dropwise. Formed solution was stirred at 90° C. (heating temp.) for 5 hours.

After cooling down to room temperature, 20 ml isopropyl ether and 20 ml water were added. Mixture was stirred for 20 minutes. Separated aquous layer was neutrallized to pH 8, by addition of a 2 N NaOH solution. Mixture was extracted with ethyl acetate (2×20 ml). Combined ethyl acetate layer was washed with H₂O, brine, dried and concentrated to give an oily material. The oil was re-dissolved in 10 ml dried toluene. After partly evaporated on rotorvapor, solid was precipitated. The solid was filtered off.

960 mg of a solid product was obtained after drying at 40° C. in vacuo over night.

### Example 8

### Step 1—Compound (7a)

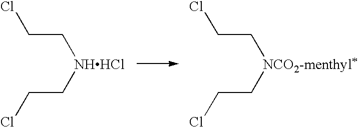

To a suspended bis(chloroethyl)amine HCl salt (5.0 g) in 30 ml dry dichloromethane, with cooling (ice water) and stirring, (−)-menthyl chloroformate (6.4 ml) was added dropwise. The addition was completed within 20 min followed by addition of triethylamine (8.9 ml) in 55 min. The mixture was further stirred at room temperature for 30 min. Water (10 ml) was added, and the mixture was stirred for 20 min. Separated dichcloromethane layer was washed with HCl 1M(10 ml), brine (10 ml), dried and concentrated in vacuo to give an oily product (7.5 g, ˜83% yield).

### Step 2—Compound (8a)

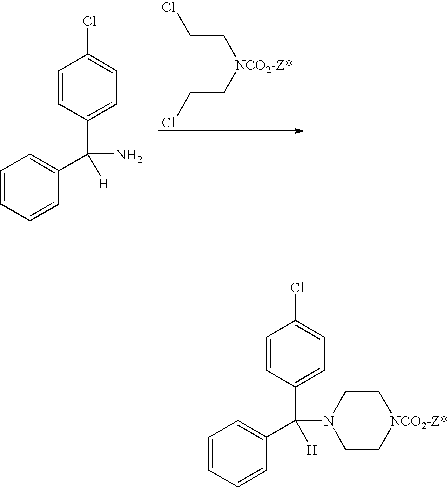

A mixture containing (4-chlorophenyl)phenyl methylamine hydrochloride salt (4.1 g), bis(chloroethyl)amine menthyl carbamate (7.0 g), potassium iodide (1.7 g) and diisopropyl ethylamine (10 ml) was stirred at 120° C. (140° C. oil bath) for 6 hours.

After cooling down, 60 ml dichloromethane was added. Mixture was stirred at ambient temperature for 30 min. and organic layer was separated. The separated organic layer was washed with HCl solution (1M, 30 ml) and brine (20 ml). It was dried and concentrated to give an oily material (10 g).

### Step 3—Resolution of the Compound (8a)

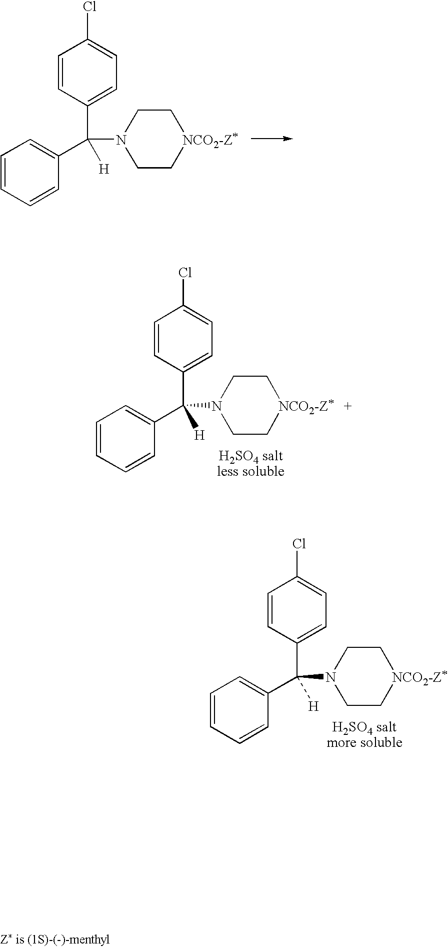

Above crude material was dissolved in ethyl acetate (40 ml). With stirring at room temperature, sulfuric acid (0.88 g) was added dropwise and sticky solid was appeared. The stirring was stopped for a while, and the supernatant was decanted.

The sticky solid was triturated in diisopropylether (10 ml) over night. Solid was filtered off, which was ˜5.5 g in total, after drying.

The solid was suspended in a mix-solvent (10 ml acetonitrile and 60 ml ethyl acetate) and stirred with refluxing for ˜1 hour. Solid was filtered off. The washing process was repeated twice. Desired salt was obtained (1.1 g, enantiomeric purity is >97.1%).

Each of the patents, patent applications, and journal articles mentioned above are incorporated herein by reference. The invention having been described it will be obvious that the same may be varied in many ways and all such modifications are contemplated as being within the scope of the invention as defined by the following claims.

1. A compound of formula (8) or a salt thereof: 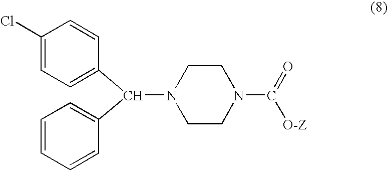
wherein Z represents a group containing 1-20 carbon atoms and at least one chiral carbon atom and having a single conformation.

2. The compound according to claim 1, wherein the chiral methyl group is in a single conformation.

3. The compound according to claim 2, wherein the chiral methyl group is in the (R)-conformation.

4. The compound according to claim 1, wherein Z is a substituted or unsubstituted alkyl group, cycloalkyl group, aralkyl group, or alkylaryl group, wherein said substituents are selected from alkyl, cycloalkyl, halogen, alkoxy, amino, and/or nitro groups.

5. The compound according to claim 4, wherein Z is a C6 to C20 substituted cycloalkyl group.

6. The compound according to claim 5, wherein said compound is a compound of formula (8a): 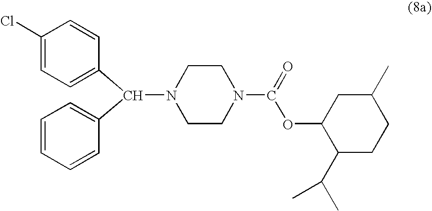
or a salt thereof.

7. The compound according to claim 6, wherein said compound is a sulfate salt.

8. A process, which comprises:
(a) providing a pair of diastereomers of the formula (8) or a salt thereof: 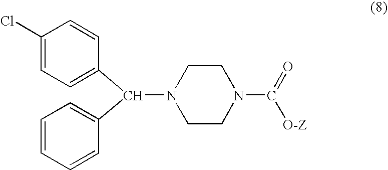
wherein Z represents a group containing 1-20 carbon atoms and at least one chiral carbon atom and having a single conformation;
(b) resolving said diastereomers of formula (8) to obtain the single diastereomer having the chiral methyl group in the R-conformation; and
(c) hydrolyzing the single diastereomer of the compound (8) having the chiral methyl group in the R-conformation to form the (R)-enantiomer of the compound (4) 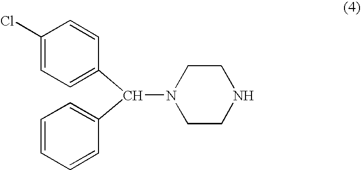
or a salt thereof.

9. The process according to claim 8, which further comprises:
(d) converting the (R)-enantiomer of the compound (4) into levocetirizine.

10. The process according to claim 9, wherein said providing step (a) comprises:
(i) reacting a racemic compound of formula (1) with a compound of formula (7) in the presence of a base, 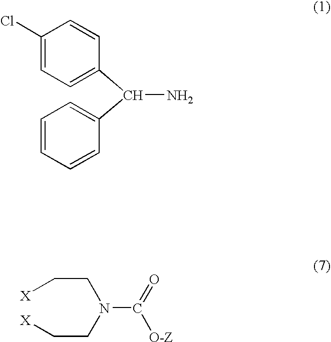
to form said pair of diastereomers of formula (8);
wherein X is a leaving group reactive with an amine; and Z is as defined in formula (8).

11. The process according to claim 10, wherein X represents a halo group or a sulfonyl group.

12. The process according to claim 11, wherein X represents a chloro, bromo, mesyloxy, besyloxy or tosyloxy group.

13. The process according to claim 12, wherein X represents a chloro group.

14. The process according to claim 9, wherein said providing step (a) comprises:
(i) reacting a compound of the formula (4) with a haloformate of the formula (9) 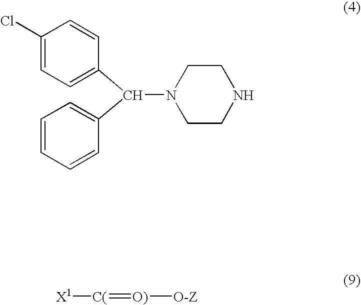
to form said pair of diastereomers of formula (8); wherein X¹ is a halo group; and Z is as defined in formula (8).

15. The process according to claim 14, wherein X¹ represents a chloro group.

16. The process according to claim 9, wherein Z represents a substituted or unsubstituted alkyl group, cycloalkyl group, aralkyl group, or alkylaryl group, wherein said substituents are selected from alkyl, cycloalkyl, halogen, alkoxy, amino, and/or nitro groups.

17. The process according to claim 16, wherein Z is a C6 to C20 substituted cycloalkyl group.

18. The process according to claim 17, wherein Z is a menthyl group.

19. The process according to claim 18, wherein Z is (−)-menthyl.
